(19) United States
(12) Patent Application Publication
(10) Pub. No.: US 2018/0202953 A1
(43) Pub. Date: Jul. 19, 2018

(54) LOW FIELD XENON CAGE RELAXOMETRY

(71) Applicant: The Regents of the University of California, Oakland, CA (US)

(72) Inventors: Muller Gomes, Berkeley, CA (US); Phuong Dao, Richmond, CA (US); Christophoros Vassiliou, Aradippov (CY); Alexander Pines, Berkeley, CA (US)

(21) Appl. No.: 15/868,074

(22) Filed: Jan. 11, 2018

Related U.S. Application Data
(60) Provisional application No. 62/447,838, filed on Jan. 18, 2017.

(51) Int. Cl. G01N 24/08; G01R 33/28; G01N 33/68

(52) U.S. Cl. CPC G01N 24/088; G01N 1/28; G01N 33/68; G01R 33/282

(57) ABSTRACT

Described herein are techniques and methods for measuring the chemical dynamics of a sample by monitoring the longitudinal and/or transverse relaxation rate of hyperpolarized xenon at low magnetic fields using a rubidium magnetometer.

### RELATED APPLICATIONS

This application claims priority to U.S. Provisional Patent Application No. 62/447,838, filed Jan. 18, 2017, which is herein incorporated by reference.

### STATEMENT OF GOVERNMENT SUPPORT

This invention was made with government support under Contract No. DE-ACO2-05CH11231 awarded by the U.S. Department of Energy. The government has certain rights in this invention.

### BACKGROUND

Hyperpolarized xenon has been used in a variety of nuclear magnetic resonance (NMR) applications. However, all current techniques require the use of a large external magnetic field. This limits the applicability of hyperpolarized xenon NMR because large magnets are expensive and difficult to transport.

### SUMMARY

Described herein are techniques and methods for detecting an analyte of interest. In some embodiments (e.g., see FIG. 4), the method comprises: contacting a sample comprising the analyte of interest with a hyperpolarized noble gas (405); applying a bias magnetic field to the sample (410); and measuring the nuclear magnetic relaxation rate of the hyperpolarized noble gas to detect a binding event between the hyperpolarized noble gas and the analyte of interest (415). In some embodiments, the nuclear magnetic relaxation rate of the hyperpolarized noble gas is measured to detect a binding event between the hyperpolarized noble gas or molecule coupled to the hyperpolarized noble gas and the analyte of interest. In some embodiments, the hyperpolarized noble gas is coupled to a molecular sensor. In some embodiments, the molecular sensor comprises a cryptophane and the hyperpolarized noble gas is encaged within the cryptophane. In some embodiments, the molecular sensor further comprises a targeting moiety. In some embodiments, the targeting moiety is biotin.

In some embodiments, the analyte of interest is a peptide chain. In some embodiments, the analyte of interest is a protein. In some embodiments, the analyte of interest comprises avidin. In some embodiments, the bias magnetic field strength is less than 1 Tesla. In some embodiments, the bias magnetic field strength is less than 15 mT. In some embodiments, the nuclear magnetic relaxation rate is measured with a rubidium magnetometer. In some embodiments, the noble gas is Xe. In some embodiments, the noble gas is ¹²⁹Xe. Moreover, in some embodiments, the noble gas is He. In some embodiments, the noble gas is ³He.

In some embodiments, the nuclear magnetic relaxation rate is a longitudinal relaxation rate. In some embodiments, the nuclear magnetic relaxation rate is measured by applying a DC magnetic field pulse orthogonal to the bias magnetic field. In some embodiments, the magnetic field is Earth's magnetic field.

### BRIEF DESCRIPTION OF THE DRAWINGS

FIG. 1 shows a plot of the relaxation rate of xenon inside a cryptophane cage. As the correlation time increases, the relaxation rate increases as well. The effect is most pronounced at low magnetic fields.

FIG. 2 shows the slope of the relaxation rate of xenon as a function of correlation time converges at 10 mT.

FIG. 3 shows an example apparatus for use in the techniques and methods described herein.

FIG. 4 shows an example of a flow diagram illustrating a method of detecting an analyte of interest.

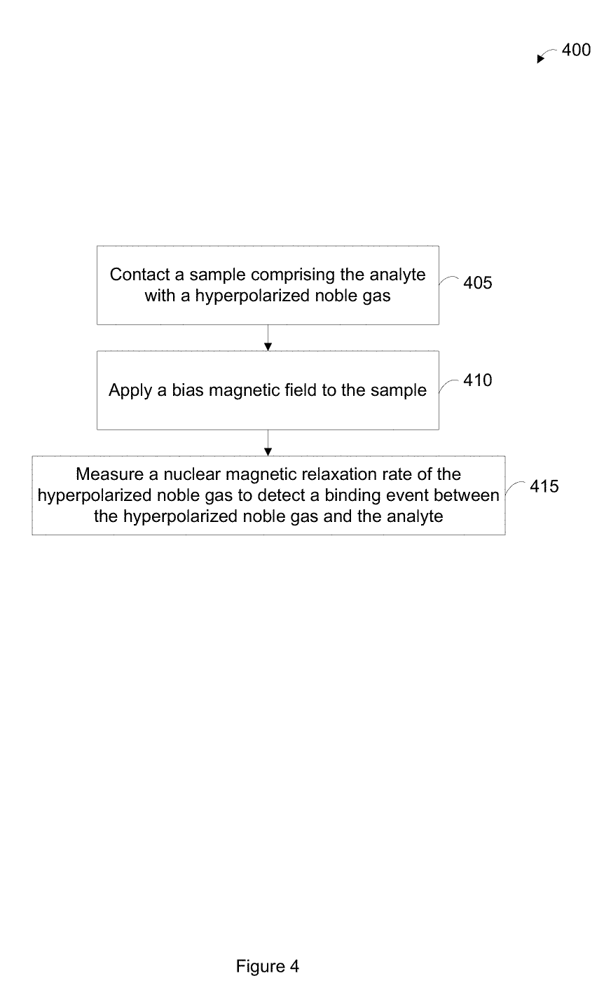

### DETAILED DESCRIPTION

Xenon relaxometry has many potential applications in drug binding assays, protein assays, or any method that requires sensitive detection of an analyte in solution. The techniques and methods described herein leverage hyperpolarized xenon relaxometry to analyze the chemical dynamics of a sample containing a compound of interest. By eliminating the need for large magnets, the techniques described herein provide significant advances in terms of accuracy, sensitivity, expense, ease of use, and portability. Moreover, by performing the measurements at low fields, changes in rotational correlation time elicit a more pronounced effect on xenon's relaxation rate. These advances allow hyperpolarized xenon NMR to expand into new areas such as highly sensitive miniaturized assays and portable devices. Furthermore, the methods and techniques described herein can be used to detect large targets, such as proteins, antibodies, and other biological molecules.

In some embodiments, the methods described herein utilize hyperpolarized xenon at low magnetic fields for chemical and biological analysis. In other embodiments, different or additional hyperpolarized noble gasses may be used, including: ¹H, ³He, ¹³C, ⁸³Kr, ¹³¹Xe and mixtures thereof. Using the techniques and methods described herein, it is possible to detect the presence or absence of chemical analytes of interest within a solution. For instance, in some embodiments, hyperpolarized xenon is bubbled into a liquid sample and allowed to relax. The dynamics of the sample, including the presence or absence of chemical analytes of interest, can alter the rotational correlation time of xenon upon binding due to the difference in mass between xenon and the xenon-target complex. The change in the rotational correlation time of xenon will alter the rate at which hyperpolarized xenon relaxes to its ground state. Accordingly, by measuring these changes in the nuclear magnetic relaxation time of xenon, it is possible to monitor changes in the chemical or biological composition of the sample and detect xenon-binding events.

Hyperpolarization of xenon, or other appropriate species, may be achieved through a variety of techniques known to those skilled in the art. One possible hyperpolarization technique is spin-exchange optical pumping. This process utilizes a circularly polarized laser, tuned to a transition frequency of an alkali vapor, to excite and spin-polarize the electron spin of vaporized alkali metal atoms. Rubidium is commonly used, but other alkali metals are suitable. Subsequent spin-exchange collisions between the polarized alkali metal atoms and xenon transfer the electron spin polarization of the alkali metal vapor to the nuclei of xenon, thereby producing hyperpolarized xenon. Other species suitable for hyperpolarization in accordance with the techniques and methods described herein, include ¹H, ³He, ¹³C, ⁸³Kr, and ¹²⁹Xe, among others. Hyperpolarized gasses may be introduced into a test solution via bubbling, infusion cartridges, membrane infusion, or any other convenient means. In some embodiments, hyperpolarized xenon is bubbled into a test solution using an infusion cartridge, and the flow of xenon is halted prior to performing a measurement.

As hyperpolarized xenon gas decays back to thermal equilibrium, the magnetic field it produces also decays. As described above, the rate of decay can be monitored to determine if a binding event has occurred.

Xenon is able to bind targets directly or indirectly. In some embodiments, xenon's natural affinity for amino acids and proteins allow xenon to bind directly to a target. Alternatively, in some embodiments, xenon may first be coupled to a molecular sensor, which then binds the target. For instance, in some embodiments, xenon may be encaged within a cryptophane cage. Suitable cryptophane cages include Cryptophane-A, Cryptophane-A3, Cryptophane-C, Cryptophane-D, Cryptophane-E, Cryptophane-E3, Cryptophane-F, Cryptophane-O, Cryptophane-O3, Cryptophane-δ, Cryptophane-γ, Cryptophane-223, Cryptophane-224, and Cryptophane-233, among others. In some embodiments, xenon may be introduced into a solution comprising a cryptophane-based sensor, where xenon will enter and bind the cryptophane cage. In some embodiments, the molecular sensor includes a targeting moiety coupled to a cryptophane cage. For instance, amino acid chains can be covalently attached to the cryptophane cage, and targeting moieties can be covalently attached to amino acid side chains to improve the binding affinity of the sensor to the target compound. In this manner, the sensitivity and accuracy of the detection scheme can be greatly increased. For example, in some embodiments, a cryptophane sensor can be covalently bound to an amino acid chain functionalized with biotin. The high binding affinity of biotin for avidin allows the construct to function as an effective avidin sensor. Regardless of the mechanism, any binding event between a molecular sensor coupled to hyperpolarized xenon will alter the rotational correlation time of xenon due to the increase in mass of the xenon-target complex. For instance, in some embodiments, the cryptophane-based sensor described above is about 2 kD, compared to the avidin-bound sensor having a mass of about 66 kD. Upon binding, the increase in mass of the overall complex leads to a change in the rotational correlation time of xenon, thereby altering xenon's magnetic nuclear relaxation rate. Accordingly, the chemical dynamics of the solution can be monitored by measuring the nuclear magnetic relaxation rate of xenon to determine if a binding event has occurred.

Any binding event that alters the rotational correlation time of xenon will alter the nuclear magnetic relaxation rate of xenon. As such, any analyte of interest or target having a suitably large mass, and sufficient binding affinity for xenon, or a xenon-sensor complex may be used. For instance, xenon has a natural affinity for amino acids, and will readily bind peptide chains. In some embodiments, analytes of interest may include peptides having an amino acid length of about 8 amino acids, about 10 amino acids, about 20 amino acids, between about 20 amino acids and about 40 amino acids, between about 40 amino acids and about 100 amino acids, between about 100 amino acids and about 200 amino acids, or greater than about 200 amino acids. In some embodiments, the analyte of interest may include a compound having a mass of at least about 1 kD, between about 2 kD and about 4 kD, between about 4 kD and about 10 kD, between about 10 kD and about 20 kD, between about 20 kD and about 50 kD, between about 50 kD and about 100 kD, or greater than about 100 kD.

It has been found that changes in rotational correlation time can have more drastic effects on the transverse nuclear magnetic relaxation rate (T2) than the longitudinal nuclear magnetic relaxation rate (T1). These effects can be more pronounced at lower magnetic fields. Accordingly, in some embodiments, the nuclear magnetic relaxation rate of xenon is measured at low magnetic fields using a rubidium magnetometer. Rubidium magnetometers can monitor the magnetization directly, and need not rely on a precessing magnetic moment, removing the need to use small flip angle pulse sequences to measure xenon T1 relaxation times. Rubidium magnetometers are also more sensitive than traditional detection means at low fields. While it is also possible to capture some of this sensitivity at comparatively high fields by measuring T2, T2 is also subject to chemical exchange, which can distort the measurement.

In various embodiments, detection of the xenon nuclear magnetic relaxation rate (e.g., longitudinal nuclear magnetic relaxation rate) is conducted using low magnetic field strength. For instance, in some embodiments, NMR machines utilizing low field magnets are used to determine the longitudinal relaxation rate (T1) of xenon. It is also possible to monitor the magnetization of xenon with an inductive coil by applying a DC pulse orthogonal to the bias field and then allowing the xenon to precess about a small external magnetic field. Similarly, in some embodiments, it can be advantageous to allow the hyperpolarized noble gas to relax within a relatively high field region of the magnetic field, before the sample is shuttled to a relatively low field region where changes in rotational correlation time can be more easily detected. In some embodiments, it can be advantageous to tune the inductive coil to the resonance frequency of the hyperpolarized noble gas in the high field region where the sample was allowed to relax. In some embodiments, low field magnets can be used to generate magnetic fields on the order of earth's field. In various embodiments, the magnetic field strength is lower than 3 Tesla, lower than 2 Tesla, lower than 1 Tesla, between 1 and 2 Tesla, or about 1.1 Tesla. In some embodiments, lower magnetic fields can be used. For instance, in some embodiments the magnetic field strength can be lower than 800 mT, lower than 500 mT, lower than 250 mT, lower than 100 mT, lower than 80 mT, lower than 50 mT, lower than 25 mT, lower than 10 mT, between 1 mT and 20 mT, between 5 mT and 20 mT, or about 10 mT. Low field magnets can similarly be used to generate magnetic fields in the micro Tesla range. For instance, in some embodiments, the magnetic field strength is lower than 1,000 μT, lower than 750 μT, lower than 500 μT, lower than 250 μT, lower than 150 μT, lower than 100 μT, lower than 80 μT, lower than 50 μT, lower than 25 μT, lower than 10 μT, or lower than 5 μT. In other embodiments, Earth's magnetic field is used directly. When utilizing low magnetic field strengths, the T1 relaxation rate can be determined by using low field magnetometers, such as an atomic vapor magnetometer, such as a rubidium magnetometer, or a SQUID magnetometer, to directly measure the magnetic field.

It is also possible to monitor a xenon binding event at high fields by measuring either the T2 or T1 of xenon. T1 changes much less upon binding than T2, partially because T1 is insensitive to changes in the rotational correlation time at high magnetic fields. At lower fields, T1 is much more sensitive to changes in dynamics because T1 is only sensitive to changes in molecular tumbling. Conversely, T2 is affected by a wide range of molecular interactions, including chemical shift, which can potentially obfuscate changes in the chemical dynamics of the sample. In some embodiments, NMR machines utilizing high magnetic fields are used to determine one or both of the transverse relaxation rate (T2) and longitudinal relaxation rate (T1) of xenon. In various embodiments, the magnetic field strength is higher than 6 Tesla, higher than 8 Tesla, higher than 10 Tesla, between 4 and 12 Tesla, between 8 and 11 Tesla, between 9 and 10 Tesla, or about 9.4 Tesla.

Moreover, in some embodiments utilizing high fields, at least one of T1 or T2 can be detected by leveraging field shuttling techniques. For instance, where comparatively high fields are employed, the hyperpolarized noble gas can be shuttled into a low field region where the sample is allowed to relax. Then, the sample can be shuttled to a high field region where the relaxation rate of the sample can be determined using, e.g., an inductive coil tuned to the resonance frequency of the hyperpolarized noble gas in the higher field region. In this manner, the relaxation time of the hyperpolarized noble gas can be determined in accordance with the techniques and methods described herein, even where low field magnets are unavailable or inconvenient to employ.

In some embodiments, T2 is measured by using a CPMG pulse sequence. For example, an initial 90 degree excitation pulse is applied followed by a series of 180 degree pulses that are out of phase with the excitation pulse. The resulting echoes are detected and the decay in the signal is measured to determine T2 (e.g., by fitting the signal decay to an exponential function). In some embodiments, the 180 degree pulses are repeated until the magnetization of xenon has decayed substantially. In various embodiments, the time between 180 degree pulses can be less than 400 ms, less than 300 ms, less than 250 ms, less than 100 ms, less than 50 ms, less than 25 ms, less than 10 ms, less than 5 ms, less than 1 ms, or about 0.5 ms. In some embodiments, the time between 180 degree pulses can be between 200 ms and 400 ms, between 100 ms and 300 ms, between 150 ms and 250 ms, between 0.5 ms and 10 ms, about 200 ms, about 150 ms, about 100 ms, about 50 ms, about 25 ms, about 10 ms, or about 1 ms. It is also possible to measure T1 at high magnetic fields, by delivering a series of 20 degree pulses and measuring the rate of decay in the detected signal. In some embodiments, alternate small angles may also be used. However, when the angle becomes too small, noise may overwhelm the signal, making measurements difficult. In some embodiments, the pulse angle may be less than 20 degrees, less than 18 degrees, less than 15 degrees, or less than 10 degrees. In some embodiments, the pulse angle may be between 10 degrees and 20 degrees. In some embodiments, the pulse angle may be about 18 degrees.

It will be appreciated that the T1 or T2 relaxation of xenon may be measured using any of a variety of techniques that are known in the art. The methods herein are not limited by the specific techniques described above.

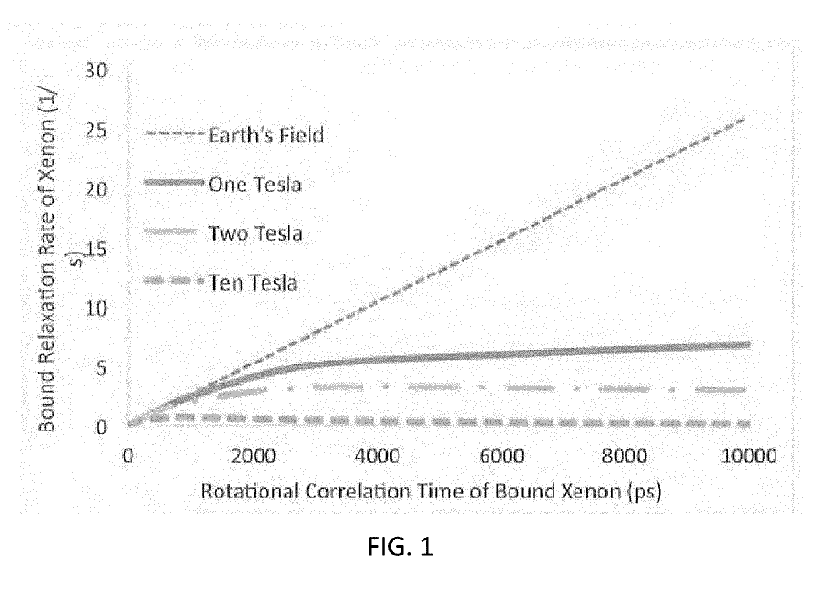

FIG. 1 is a graph depicting a simulation of the relaxation rate of xenon inside a cryptophane cage at various magnetic field strengths. The graph was generated in accordance with Equation 1, below, by simulating xenon magnetization at various fields using a MATLAB code package called Spinach.

$$\frac{1}{{T}_{1}} = \frac{2}{15} * {b}^{2} * ( \frac{τ}{1 + {( {ω}_{S} - {ω}_{I} )}^{2} {τ}_{c 2}^{2}} + \frac{3 τ}{1 + {ω}_{I}^{2} {τ}_{c 1}^{2}} + \frac{6 τ}{1 + {( {ω}_{S} + {ω}_{I} )}^{2} {τ}_{c 2}^{2}} ) \qquad Equation 1$$

As FIG. 1 shows, the change in T1 can be more pronounced at low magnetic fields. Accordingly, in some embodiments, it is advantageous to monitor the rotational correlation time of xenon at low magnetic fields. It is also possible to capture some sensitivity at high fields by measuring T2. However, T2 is also sensitive to chemical exchange. This chemical exchange can be the same after binding, which can reduce the change in T2 upon binding. If the exchange contribution is strong enough, it could eliminate the correlation time contribution. Accordingly, in some embodiments, it is advantageous to measure a value that depends solely on correlation time, thereby increasing the change in relaxation time upon binding and simultaneously increasing the sensitivity of the assay.

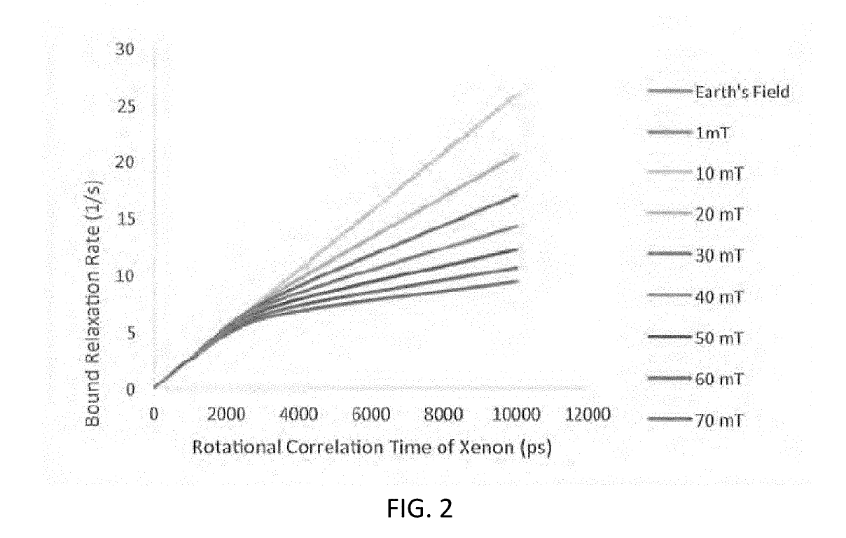

FIG. 2 is a graph depicting a simulation of the slope of xenon's relaxation rate as a function of correlation time at various magnetic field strengths, and was prepared in the same manner as FIG. 1. As FIG. 2 shows, the relaxation rate slope of xenon converges at 10 mT. Although lower fields can be used, lower fields may not improve relaxation contrast. However, there may be some exceptions. For instance, near-zero fields may prove advantageous when coupled with a spin exchange relaxation-free (SERF) magnetometer, due to the increased sensitivity afforded by the SERF regime. Similarly, such low fields may further prove to be advantageous when combined with different analytic reagents. In some embodiments, the analytic reagent is ¹²⁹Xe, and the T1 is measured at 10 mT or below. At low fields, the correlation time behavior of T2 is identical to T1. The chemical exchange contribution to T2 is also significantly diminished at low fields. For instance, at Earth's field, the chemical exchange contribution to T2 is unlikely to be noticeable. T2 is also sensitive to gradients at low fields. These gradients can be overcome by quick CPMGS, or by measuring T1 instead.

In various embodiments of the techniques and methods described herein, the presence or absence of a chemical analyte of interest in a test sample, which may be a solution, can be detected by monitoring the relaxation time of hyperpolarized xenon. In some embodiments of the techniques and methods described herein, hyperpolarized xenon is bubbled into a test solution. Hyperpolarized xenon can be generated in a rubidium cell polarizer, and bubbled into solution through a membrane infusion cartridge, or other suitable means. In some embodiments, the test solution may comprise a molecular sensor, such as a cryptophane sensor. In some embodiments, a molecular sensor may be added to the test solution after the hyperpolarized xenon has been bubbled into solution. Alternatively, in some embodiments, no molecular sensor is used and xenon is allowed to bind directly to the target compound. Once the hyperpolarized xenon has been bubbled into solution, one or both of the longitudinal or transverse relaxation time of the hyperpolarized xenon can be measured in whatever manner is most convenient for the user to establish the nuclear magnetic relaxation rate of xenon. In some embodiments, the relaxation rate of hyperpolarized xenon is measured with a rubidium magnetometer, although other means are available. A chemical analyte of interest can then be added to the solution and xenon is allowed to bind to the target compound, either directly or indirectly via a molecular sensor. The relaxation time of xenon can be monitored to detect a change in the relaxation time of xenon, signifying that a binding event has occurred.

In some embodiments, the techniques and methods described herein may be performed in vivo. For instance, xenon's change in rotational correlation, caused by a binding event between the xenon (or xenon-cage complex) and its target, can be detected via MRI, enabling a wide scope of potential applications. Accordingly, xenon may be administered to subjects alone, or in conjunction with a cryptophane cage, and the subject can be monitored via MRI to detect the occurrence of binding events. The cryptophane cage may be further functionalized with targeting moieties in order to tune the complex to identify a variety of biomarkers and biological events. For instance, certain proteins can be detected by leveraging xenon's natural affinity for amino acids, or by configuring the side chains of a cryptophane sensor to bind proteins of interest. One such example would be the synthesis of a biotin side chain on a cryptophane sensor. Due to biotin's high affinity for avidin, such a complex would form an effective avidin sensor.

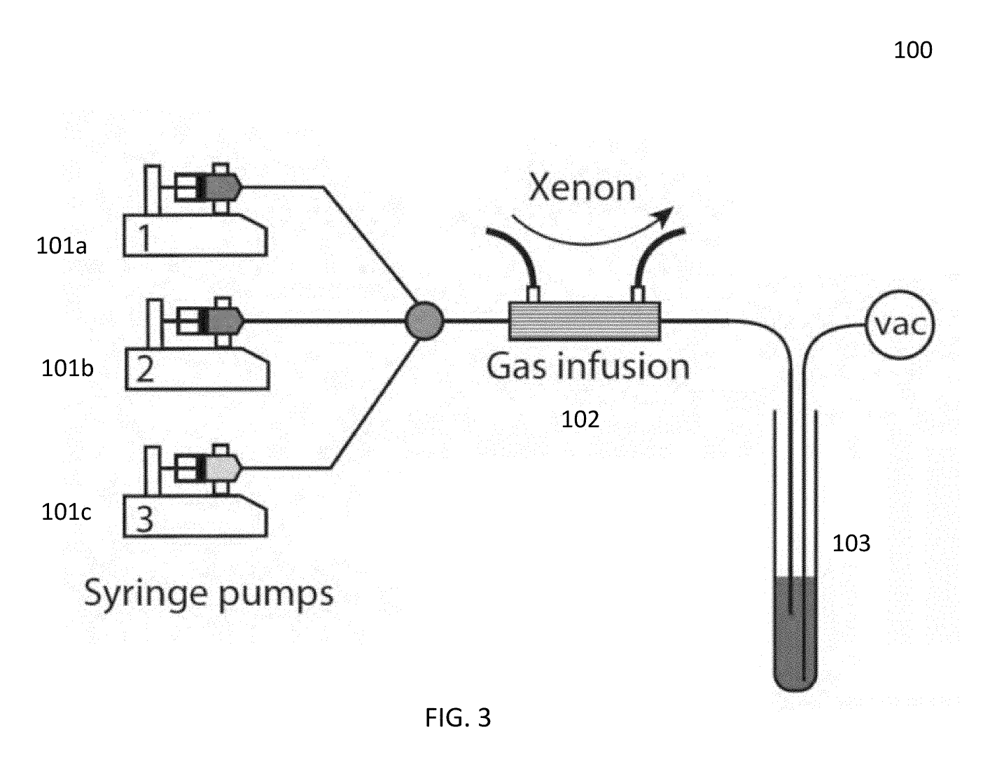

Some embodiments include an apparatus for measuring the relaxation rate of xenon. FIG. 3 depicts an example of an apparatus suitable for employing the techniques and methods disclosed herein. FIG. 1 depicts a plurality of syringe pumps 101 in fluid communication with a gas infusion cartridge 102 and test tube 103. In some embodiments, the test tube 103 may reside within a rubidium magnetometer, NMR machine, or other magnetic field detection means. In some embodiments, one syringe of the plurality of syringes 101 may contain a test solution. In some embodiments, the test solution contained in at least one syringe of the plurality of syringes 101 may comprise a molecular sensor. A second syringe of the plurality of syringes 101 may contain an analyte of interest. In some embodiments, at least one of the plurality of syringes is used to pass the test solution through an infusion cartridge 102. The gas infusion cartridge 102 allows the operator to bubble in the desired concentration of hyperpolarized gas. The solution may then be injected directly into a suitable container, such as a test tube 103 residing in a rubidium magnetometer, or other magnetic field detection means. In some embodiments, hyperpolarized xenon can be infused from a rubidium polarizer external to a rubidium magnetometer. It is thus possible to measure the relaxation time of the hyperpolarized gas immediately, or to inject a chemical analyte of interest prior to performing a relaxation time measurement.

While an apparatus comprising two-pumps as described above may be appropriate for studying interactions between xenon and target compounds, additional pumps may be added. Examples include adding a second or third drug to investigate interactions at the protein level, adjusting the solution pH, or changing salt concentrations. However, additional chemical environments can be studied as well. The parameters and contents of each syringe may vary based on the chemical environment of interest.

What is claimed is:

1. A method of detecting an analyte, the method comprising:
contacting a sample comprising the analyte with a hyperpolarized noble gas;
applying a bias magnetic field to the sample; and
measuring a nuclear magnetic relaxation rate of the hyperpolarized noble gas to detect a binding event between the hyperpolarized noble gas and the analyte.

2. The method of claim 1, wherein the hyperpolarized noble gas is coupled to a molecular sensor.

3. The method of claim 2, wherein the molecular sensor comprises a cryptophane, and wherein the hyperpolarized noble gas is encaged within the cryptophane.

4. The method of claim 3, wherein the molecular sensor further comprises a targeting moiety.

5. The method of claim 4, wherein the targeting moiety is biotin.

6. The method of claim 1, wherein the analyte is a peptide chain.

7. The method of claim 1, wherein the analyte is a protein.

8. The method of claim 1, wherein the analyte comprises avidin.

9. The method of claim 1, wherein the bias magnetic field strength is less than 1 Tesla.

10. The method of claim 1, wherein the bias magnetic field strength is less than 15 mT.

11. The method of claim 1, wherein the nuclear magnetic relaxation rate is measured with a rubidium magnetometer.

12. The method of claim 1, wherein the noble gas is Xe.

13. The method of claim 1, wherein the noble gas is ¹²⁹Xe.

14. The method of claim 1, wherein the noble gas is He.

15. The method of claim 1, wherein the noble gas is ³He.

16. The method of claim 1, wherein the nuclear magnetic relaxation rate is a longitudinal relaxation rate.

17. The method of claim 16, wherein the nuclear magnetic relaxation rate is measured by applying a DC magnetic field pulse orthogonal to the bias magnetic field.

18. The method of claim 1, wherein the magnetic field is Earth's magnetic field.
